(19) United States
(12) Patent Application Publication
(10) Pub. No.: US 2023/0011168 A1
(43) Pub. Date: Jan. 12, 2023

(54) Bio-Chips and Production Method Thereof

(71) Applicants: China Medical University, Taichung City (TW); China Medical University Hospital, Taichung City (TW)

(72) Inventors: Yi-Wen Chen, Taichung City (TW); Ming-You Shie, Taichung City (TW); Chien-Chang Chen, Taichung City (TW)

(21) Appl. No.: 17/508,140

(22) Filed: Oct. 22, 2021

(30) Foreign Application Priority Data
Jul. 9, 2021 (TW) 110125221

(51) Int. Cl. G01N 33/50; C12M 1/34; C12M 1/42; B01L 3/00; C12N 5/071; B33Y 80/00; B33Y 10/00; B33Y 40/20; B29C 64/35; B29C 64/10

(52) U.S. Cl. CPC G01N 33/5082; C12M 41/46; C12M 35/08; B01L 3/502723; B01L 3/502761; C12N 5/0691; B33Y 80/00; B33Y 10/00; B33Y 40/20; B29C 64/35; B29C 64/10; B01L 2300/0819; B01L 2200/0684; B01L 2200/0647; B01L 2300/0877; B01L 2200/12; B29K 2105/0035

(57) ABSTRACT

The present invention is related to a biochip and production method thereof. The biochip comprises a carrier, a cell or tissue culture area deposited on the carrier, and a sensor area deposited on the carrier adjacent and fluidly communicating with the cell or tissue culture area. A containing space is contained in the cell or tissue culture area comprising a simulated vascular channel, a cell or a tissue and a culture medium. At least one sensor fixation area is contained at the sensor area for placing a sensor element. The present invention can be a model for stimulating cancer of specific patient to realtimely reflecting the cancer formation, transferring status and treatment strategies. The biochip could also carry testing drugs to observe how the drugs functioning to the cells/tissue as to provide a more accurate instruction of the drugs. The present invention can perform multiple test just within on chip which can save cost and also provide a more accurate test model for the patient.

### FIELD OF INVENTION

Present invention is related to a simulation model in a field of regenerative medicine, in particular, to the simulation model in a form of biochip for bionic simulation of cells, tissues or even organ in-vivo environment.

The biochip of the present invention is firstly applied to several cancer simulation models, such as cervical cancer, colon cancer and lung cancer, and the following description with multiple embodiments will be described hereinafter in detail. However, the biochip of the present invention is not intended to be limited to these certain cancer applications. Other similar or equivalent alternation or applications are all covered within the scope of the claimed invention.

### BACKGROUND OF THE INVENTION

Although development of biology in the past ten decades has greatly improved and also promoted human life with more healthier approaches, large amount of biological experiments still remains at a simple transitional cell culture level. However, this over-simplified research method is not only difficult to truly reflect the complex functions of tissues and organs in the human body, but also difficult to reflect the true conditions of human tissues and organs to external stimuli.

Planar two-dimensional (2D) experimental test model has its advantage of easy to operate and more efficient in analyzing the effects of different experimental parameters. The interaction between cells to cells and cells to materials in 2D culture is not too accuracy as the real condition in vivo which always have more complicated interaction between cells, tissue and organs. When the cultured cells adapt to a 2D planar environment, it is difficult to maintain their actual cellular characterization resulting in a huge gap between the simulated test results and the actual situation.

Although animal experiments can provide more comprehensive studies and analysis of cells, tissues and organs, there are still significant deficiencies such as species differences between experimental animals and humans. As advanced countries such as the United States or the European Union are gradually banning animal experiments due to humility issue, how to provide an actual testing method has pushed the development of biochips. It has provided an innovative solution based on the level of tissues and organs to solve the old cell cultures and animal experiments.

### SUMMARY OF THE INVENTION

In order to solve the deficiencies and defects of the aforementioned prior art, the present invention aims to improve these deficiencies and defects such as the inaccuracy of cell culture and animal experiments and the poor ability to predict the actual human response. Also, more and more advanced countries like United States or European countries are gradually prohibiting the animal experimentation promoting the biochip being more popular for the studies of regenerative medicine, cancer research and other fields.

In accordance, a first concept provided by the present invention is a biochip comprising a carrier; a cell or tissue culture area deposited on the carrier; a sensor area deposited on the carrier adjacent and fluidly communicating with the cell or tissue culture area; a containing space is contained in the cell or tissue culture area comprising a simulated vascular channel, a cell or a tissue and a culture medium; and at least one sensor fixation area is contained at the sensor area for placing a sensor element.

In accordance, a second concept of the present invention is a production method of the biochip comprising steps of:

printing the biochip by three-dimensional printing;

filling the culture medium in the containing space of the cell or tissue culture area by three-dimensional printing; placing a tubular support produced by a solvent-soluble material on a surface of the culture medium, and the supply port and the discharging port is connected with the tubular support;

printing a vascular simulating material along a surface of the tubular support to form the simulated vascular channel and following with printing and distributing the cells and the culture medium in the rest of the containing space covering the simulated vascular channel; and

using a solvent able to dissolve the solvent-soluble material to wash off the tubular support for the containing space to obtain the biochip.

According to the above description, the advantages of the present invention are as follows.

1. The advantage of the present invention using the microphysiological system platform and 3D printing manufacturing is that the structural state and quantity of cells and tissues on the biochip can be adjusted or the position of cells and tissues on the biochip can be adjusted according to the requirement. Researchers or developers could have a better understanding of the response to drugs or nutrients of cells, tissues and organ by simulative the actual human body on the biochip. The present invention provides a more accurate simulative culture platform compared to the conventional cell culture in a culture dish (also called two-dimensional experimental test model). The present invention could be produced by mold processing which could be in a condition of better cost-competitive to the conventional testing kits or method. It also avoids to use experimental animals for humanity issue.

2. One of the multiple applications of the present invention is the research of cancer. The present invention could act as a patient cancer simulation model to reflect the cause, the potential of metastasis and also the suitable treatment for the patient. For drug screening, the biochip of the present invention could simulate a bioenvironment for reflecting how cancer cell response in human body which is a solid reference for the cancer treatment for the patient. The present invention has a great value and contribution for different stages of drug developments including drug screening, clinical phase I of drug testing, precision drug application, new drug testing, and personalized drug screening. Moreover, the biochip of the present invention could test different drugs at the same time providing a more time and cost saving test platform with a more accuracy result.

3. The biochip provided by the present invention can basically be applied three major fields. First, in medical-related industries, the biochip can be used as a tool for personalized precision test kits or test model. By using the cell directly retrieving from the patient to build the test model, the present invention could reflect a real-time response of the patient's cells by treating with different drugs for evaluating the most precisive treatment to the patient. Second, in the pharmaceutical, biotechnology, cosmetic, and chemical industries, the biochips of the present invention could also be used to build a standardized and reliable testing tools and platforms which can efficiently and instantly perform product safety or toxicology tests also avoiding the need for animal experiments. Also, the present invention could also avoid the test variation caused by the animal experiments from biological variation of the different species. Third, in academic research, it is possible to explore the path of bacteria, viruses and other infectious diseases to invade and infect human organs and tissue for new drugs development creating a test platform for the development of new drugs.

### BRIEF DESCRIPTION OF THE DRAWINGS

FIG. 1 is an illustration of a preferred embodiment of biochip in accordance to the present invention.

FIG. 2 is an illustration of using a preferred embodiment of biochip in accordance to the present invention.

FIG. 3 is a flow chart illustration of producing the preferred embodiment of biochip in accordance to the present invention.

FIG. 4 is another illustration of using a preferred embodiment of biochip in accordance to the present invention.

FIG. 5 is an illustration of cell migration in the biochip in accordance to the present invention.

FIG. 6 is an immunohistochemistry of simulated vascular channel in the biochip in accordance to the present invention.

FIG. 7 is another immunohistochemistry of the simulated vascular channel in the biochip in accordance to the present invention.

FIGS. 8A to 8C are multiple quantitative and qualitative test results using different drugs treating different cervical cancer cells.

FIG. 9 is a result of indicator protein from metabolic fluid perfusing from the biochip in accordance to the present invention.

FIGS. 10A and 10B are two immunohistochemistry of metastasis of cancer cells.

FIG. 10C is a test result indicating indicator components generated from metastasis cancer cells in the simulated vascular channel in the biochip in accordance to the present invention.

FIG. 11 is an immunohistochemistry of lung cancer cell.

FIGS. 12A and 12B are tests results of cytotoxicity tests with 5-Fu cancer cell therapeutic drug.

FIGS. 13A and 13B are cancer cell survival tests of three different cancer target drugs Erlotinib, Gefitinib and Afatinib using two different lung cancer cells HCC827 and GR10.

FIG. 13C is a cancer cell survival test of a control group of normal blood vessels corresponded to FIGS. 13A and 13B.

FIG. 14 is a validation test of cell viability of breast cancer cells treating by couples of cancer therapeutic drugs.

FIG. 15 is an immunohistochemistry corresponded to the test result of Taxol drug in FIG. 14.

### DETAILED DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts. It is not intended to limit the method by the exemplary embodiments described herein. In the following detailed description, for purposes of explanation, numerous specific details are set forth in order to attain a thorough understanding of the disclosed embodiments.

### A Preferred Embodiment of Biochip

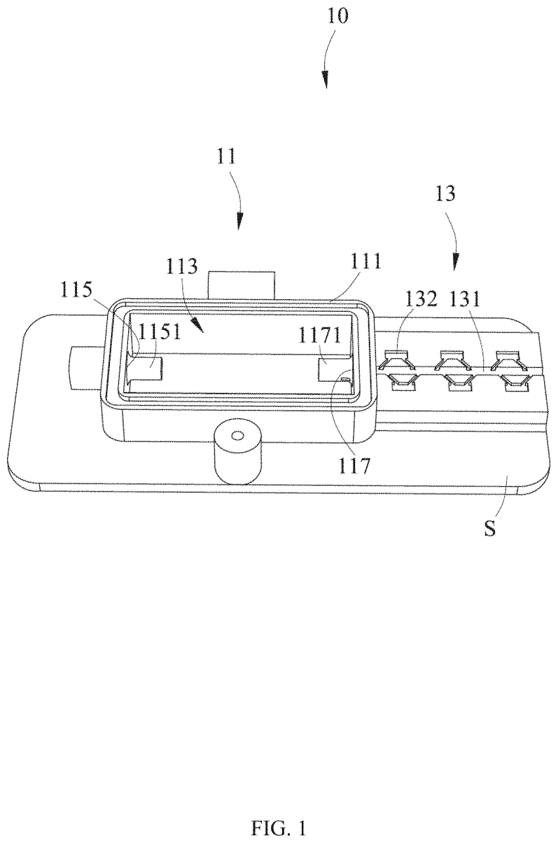

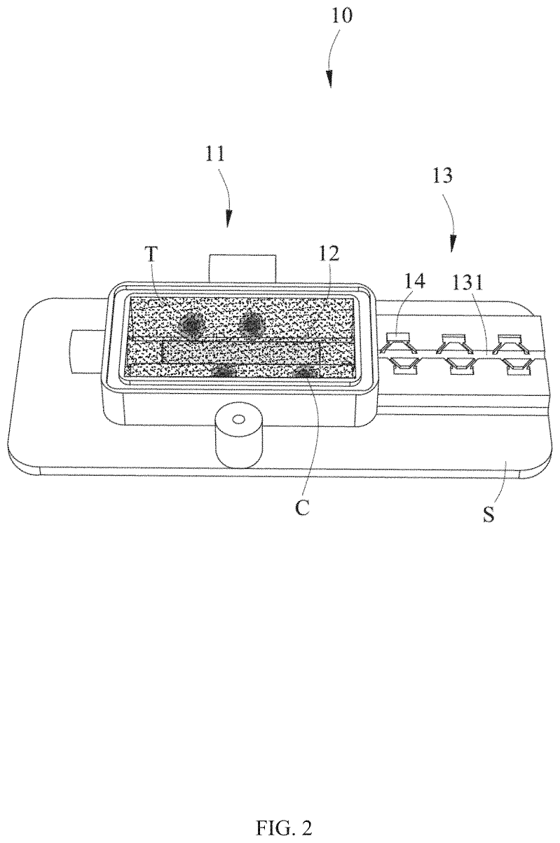

With reference to FIGS. 1 and 2, a preferred embodiment of the biochip 10 of the present invention includes a cell or tissue culture area 11 and a sensor area 13 disposed on a carrier S. Wherein, the carrier S is mainly in the shape of a flat plate, or more preferably in the shape of a long flat plate, and preferably is integrally formed and fixed with the cell or tissue culture area 11 and the sensor area 13 thereon.

The cell or tissue culture area 11 as shown in FIG. 1 is disposed on a left side of the carrier S and comprises a wall 111 that surrounded upwardly from a plane surface of the carrier S. A containing space 113 is formed inside the wall 111. A supply port 115 and a discharge port 117 are in fluid communication relation deposited on the wall 111. In this embodiment, the supply port 115 and/or the discharge port 117 are symmetrically deposited on both sides of the wall 111 against to each other, respectively. In other preferred embodiment, the supply port 115 and/or the discharge port 117 may also include a supply port connecting portion 1151 and a discharge port connecting portion 1171 protruding inwardly or outwardly from the wall 111.

In this embodiment as shown in FIG. 1, the sensor area 13 is deposited and connected with the cell or tissue culture area 11 at a right side of the carrier S. The sensor area 13 includes a fluid flow channel 131 and at least one sensor fixation area 132. In some preferred embodiments as shown in FIG. 1, several sensor fixation areas 132 are disposed in the sensor area 13. The sensor fixation area 132 may be a recess as shown in FIGS. 1 and 2 for fixing or placing a sensor element 14 in the sensor area 13. The fluid flow channel 131 and the sensor fixation area 132 are in a fluid or liquid communication with the cell or tissue culture area 11 through the discharge port connecting portion 1171.

With reference to FIG. 2, when using the biochip 10 provided in the present invention, a simulated vascular channel 12 will be formed to connect the supply port 115 and the discharge port 117. A cell C (or alternatively acceptable options like organoids, spheroids or tissues) will be filled and cultured in the containing space 113 of the cell or tissue culture area 11 within the wall 111. The said cells C are immersed in a culture medium T filled also in the containing space 113, which optionally could further contain immune cells and fibroblasts to make the cultured environment become more closed to the real biological condition. By keep supplying nutrients or even drugs and medicines to the cells C from the supply port 115, the cell C can be normally grown and metabolized in the cell or tissue culture area 11. The metabolized products will also follow the liquid flows out from the discharge port 117 into the sensor area 113 for testing or detection. The sensor area 113 is provided with the sensing element 14 according to the type of the cell C and the purpose of the testing. The metabolites will become an indicator for the sensing element 14 proving the cells C maybe response to a new drugs or medicine providing a more efficient testing solution for the validation or side effects of a new treatment.

### A Preferred Embodiment of Manufacturing the Biochip

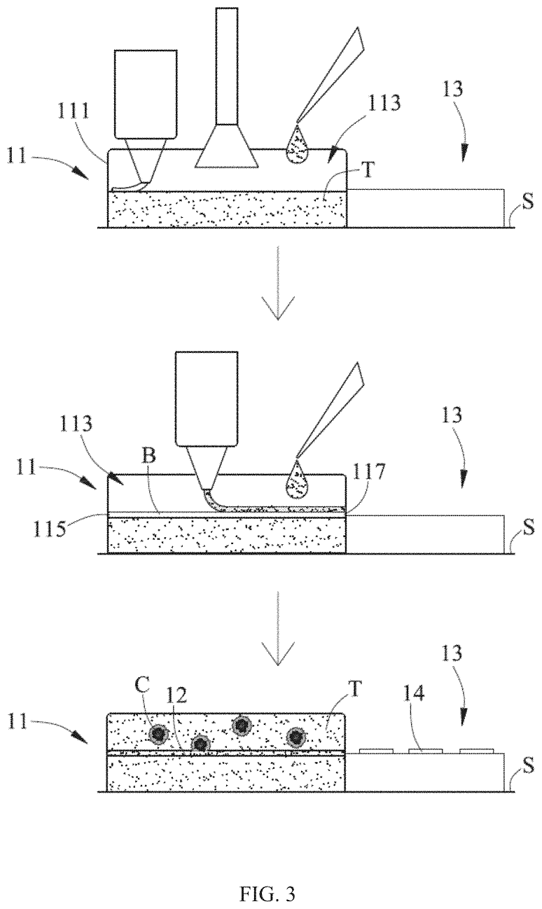

With reference to FIG. 3, a preferred embodiment for manufacturing and how to use the biochip 10 provided in the abovementioned embodiment of the present invention are presented in details. The manufacturing method comprises the steps of:

Step 1) printing the carrier 11 and the cell or tissue culture area 11 and the sensor area 13 thereon by a three-dimensional printing method (3D printing, 3D Printing), preferably a photocuring three-dimensional printing method;

Step 2) filling the culture medium T in the containing space 113 of the cell or tissue culture area 111 by also three-dimensional printing at a lower site than the height of the wall 111, better only reaches to the height of the wall 111 at the half site.

Step 3) Placing a tubular support B (or also could be a column or a long-extended bar) produced by a solvent-soluble material on a surface of the culture medium T, and the supply port 115 and the discharging port 117 is connected with the tubular support B;

Step 4) Printing a vascular simulating material along a surface of the tubular support B to form the simulated vascular channel 12 and following with printing and distributing the cells C and the culture medium T in the rest of the containing space 113 space covering the simulated vascular channel 12;

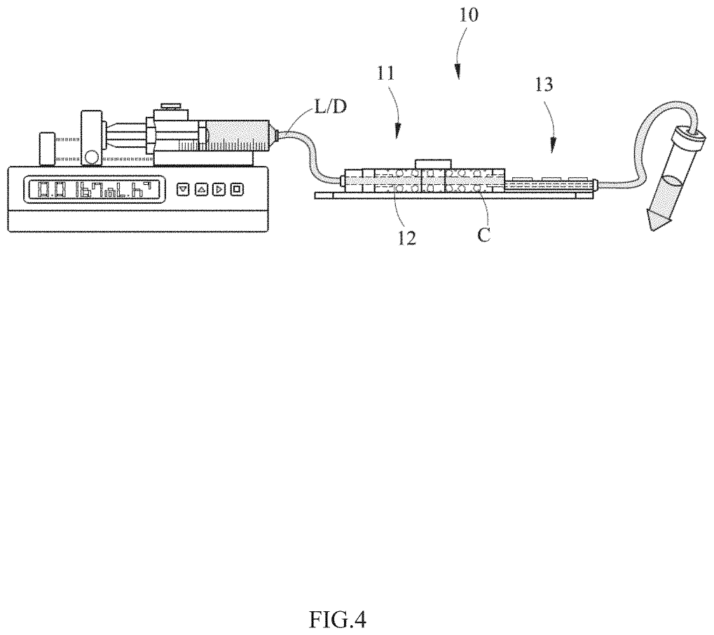

Step 5) Using a corresponded solvent able to dissolve the solvent-soluble material to wash off the tubular support B for the containing space 113, and fluxing a endothelial cell (which is a cell type forming the blood vessels) into a channel that formed by washed tubular support B to adhere, distribute and culture at an inner surface to form the simulated vascular channel (as shown in FIG. 4) to complete the assemble of the cell or tissue culture area 11 of the present invention.

Step 6) Optionally, placing the sensing element 14 according to the type of the cell C in the sensor fixation area 132 of the sensing area 13 to further complete the assembly of the biochip 10 of the present invention.

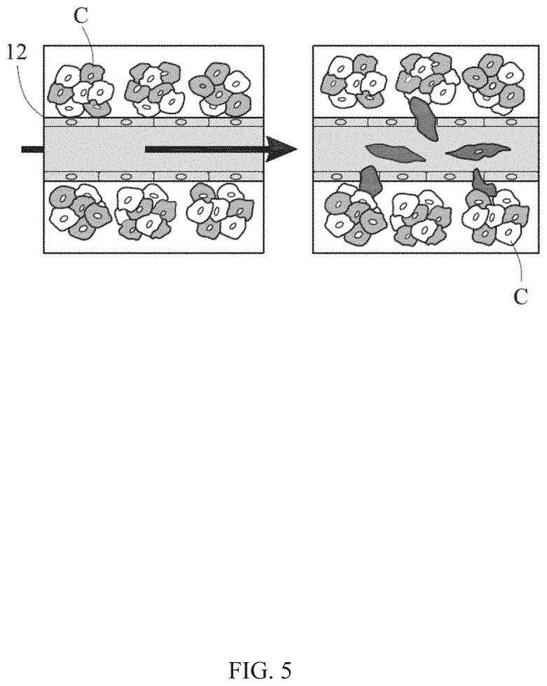

With reference to FIG. 4, the cells C in the biochip 10 produced by the manufacturing method as above needs a nutrient source liquid L and/or a drug D supplied from the outside, and the simulated vascular channel 12 permeating and supply the nutrient source liquid L and/or the drug D to the cell C. The metabolites of the cell C flow from the simulated vascular channel 12 to the sensor area 13 for the sensor element 14 detecting or testing. The detecting or testing result from the composition and characteristics of the metabolites of the cell C could be used to confirming if the supplied drug D or medicine has an expectation function or ability. On the other hand, with reference to FIG. 5, it only shows partially of the cell or tissue culture area 11 of the biochip 10 of the present invention, and the cells C may migrate to other areas through the simulated vascular channel 12 during the culture process. This could be another function of the present invention to be used as a test model for monitoring cancer cells metastasize.

On the other hand, the cells C used in the present invention can preferably be presented in a three-dimensional condition (a cell spheroid) using shape formable medium. By providing different-sized molds containing multiple recesses using three-dimensional printing, the manufacturer could just simply inject any shape formable material containing the cells C into the molds and produced the said the three-dimensional cells according to requirements. Such approach can also use in mass-production for such cell spheroid. All the materials including the biochip 10 and the cell culture medium are biocompatible materials which can effectively reduce the risk of any artificial or harmful chemical causing cell, gene (DNA) damage or affect tissue repair and regeneration ability.

### Validation Tests of Preferred Embodiments of the Biochip

The biochip 10 can be first served as a simulation model of a drug screening for cancer. This embodiment is an example of a drug screening for candidate drugs for cervical cancer taking from a target patient. It is worth mentioning that the biochip 10 provided by the present invention, the simulated vascular channel 12 in the preferred embodiment can be used for simulating an in-vivo environment of cervical cancer or breast cancer which these cancer cells are normally metabolized from the blood vessel in real human body. However, by switching the simulated vascular channel 12 into a gas or air flow channel (simulation of human trachea), it can be used as a simulation for lung cancer as another suitable applications for the present invention.

In this embodiment, when producing the simulated vascular channel 12, the fluid containing endothelial cells will be fluxed for completely adhering, attaching and even starting to be cultured to the inner surface of the simulated vascular channel 12 at 37° C. for one day. Further, the peristaltic pump is connected to circulate as a biosystem. For a single simulated vascular channel 12, it is preferred to have a perfusion rate as 13 μL/min. The present invention prefers to maintain the cells and its culture medium perfectly in a gel-like condition at 37° C., which can provide a better simulation which the target cells existing in a real human body or organs.

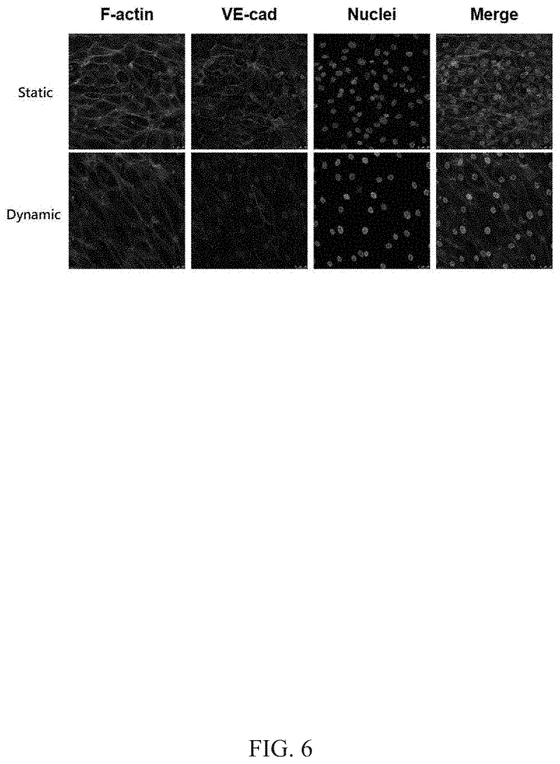

With reference to FIG. 6, it is an immunohistochemistry of the simulated vascular channel 12 under static culture condition and dynamic perfusion culture condition comprising F-actin, VE-cad, Nuclei and merge images from first three images. FIG. 6 shows the results of the static culture condition without perfusion and the dynamic culture condition with perfusion. It can be observed that the distribution of vascular cells under dynamic perfusion culture is orthotropic, and it can be clearly seen that they grow in the same direction, which is more similar to actual human bionic blood vessel growth condition.

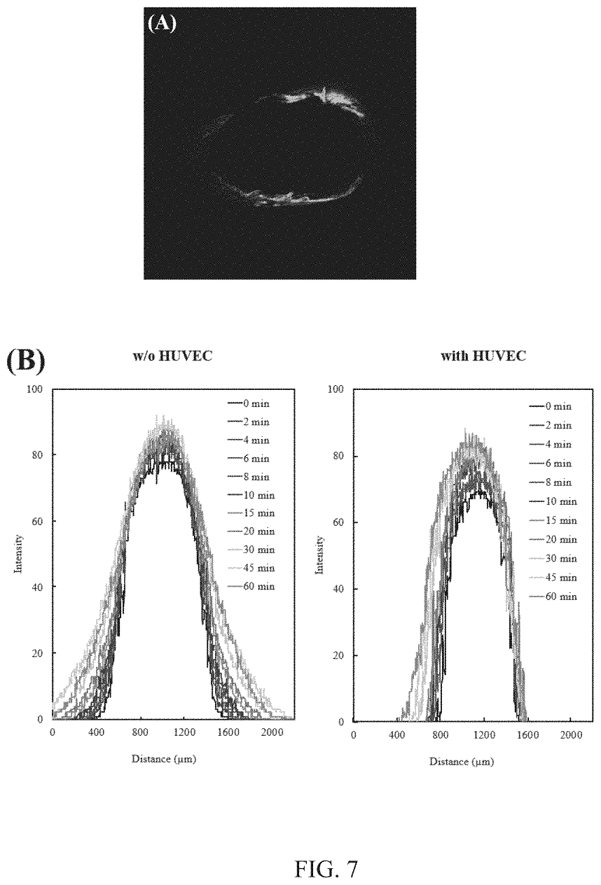

As shown in another immunohistochemistry of the simulated vascular channel 12 in left side of FIG. 7 marked in (A), it shows clearly blood vessel walls being successfully produced by the present invention. On the right side of FIG. 7 marked with (B), it presents with two images of fluorescent substances diffuses by using a control group without HUVEC (a blood vessel cell) and the present invention with HUVEC. It can be clearly seen that the control group without HUVEC on the left has a faster diffusion rate of fluorescent substances, while the present invention with HUVEC on the right side has a slower diffusion rate of fluorescent substance which is more similar to a real behavior of vascular barriers in human body.

A series of tests for characteristics and drug screening of different cancers will be performed using the biochip 10 provided by the present invention. The cancers comprise cervical cancer, lung cancer and breast cancer, respectively.

<Cervical Cancer>

When the embodiment is actually used, the cervical cancer cells or tissues sample are taken from the patient to be tested as the cell C and culturing medium T of the present invention.

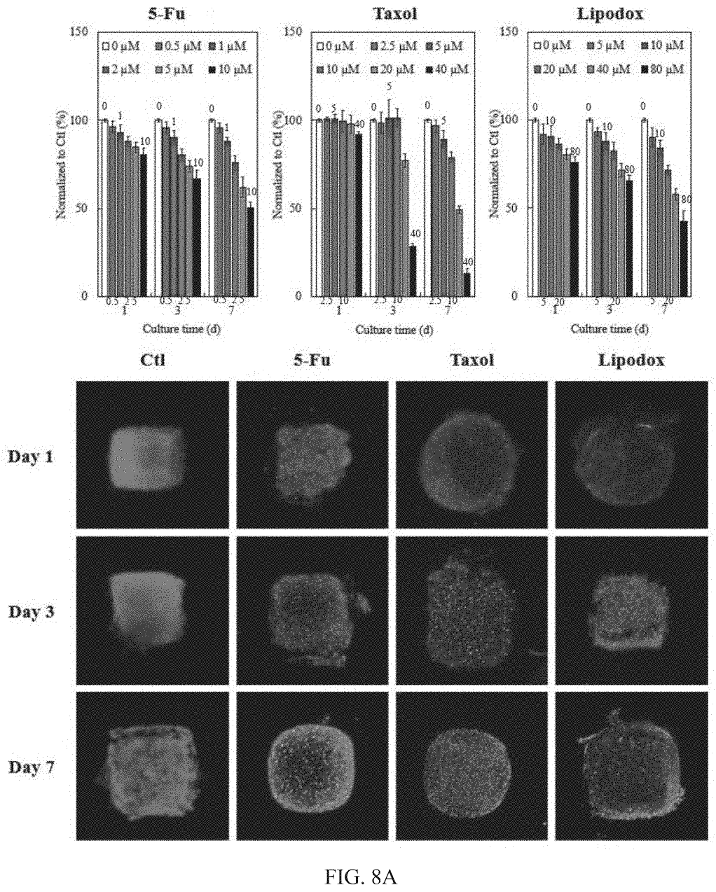

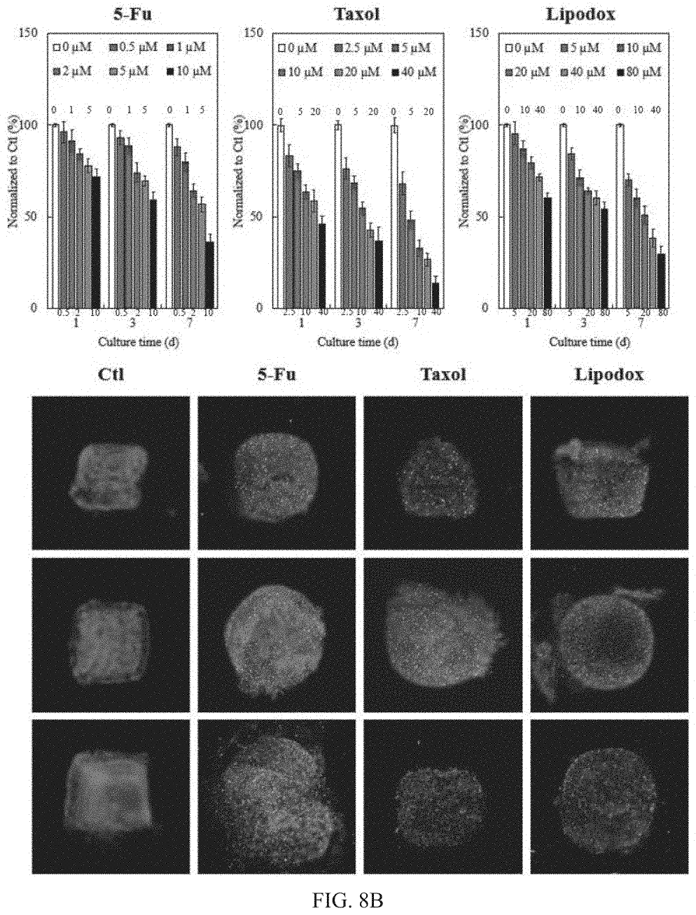

With reference to FIGS. 8A to 8C, in this embodiment, three different cervical cancer cells are used to perform quantitative and qualitative tests of the efficacy of different cancer therapeutic drugs.

FIG. 8A shows Hela cervical cancer cells reacting with three different poisoning drugs including 5-Fu, Taxol and Lipodox, respectively. Showing in the bar diagrams at the top of FIG. 8A, Taxol has a better cancer cell poisoning effect among other two drugs. As shown in the immunohistochemistry below, the lighter spots indicated the apoptotic cancer cell of tissue. The result also shows that Taxol has a significant cancer cell killing effect on the 7th day. The present invention has the ability for drugs or medicine screening for treating cancer and solve the problem of traditional cancer treatment trying the medicine on the patient one by one which may causing extra burden to the patient's body. The present invention could efficiently choose the right drugs or medicine for cancer treatment in a short time.

FIG. 8B shows SiHa cervical cancer cells reacting with three different poisoning drugs including 5-Fu, Taxol and Lipodox, respectively. Showing in the bar diagrams at the top of FIG. 8B, Taxol also has a better cancer cell poisoning effect among other two drugs.

FIG. 8C shows CCC cervical cancer cells reacting with three different poisoning drugs including 5-Fu, Taxol and Lipodox, respectively. Showing in the bar diagrams at the top of FIG. 8C, Taxol also has a better cancer cell poisoning effect among other two drugs.

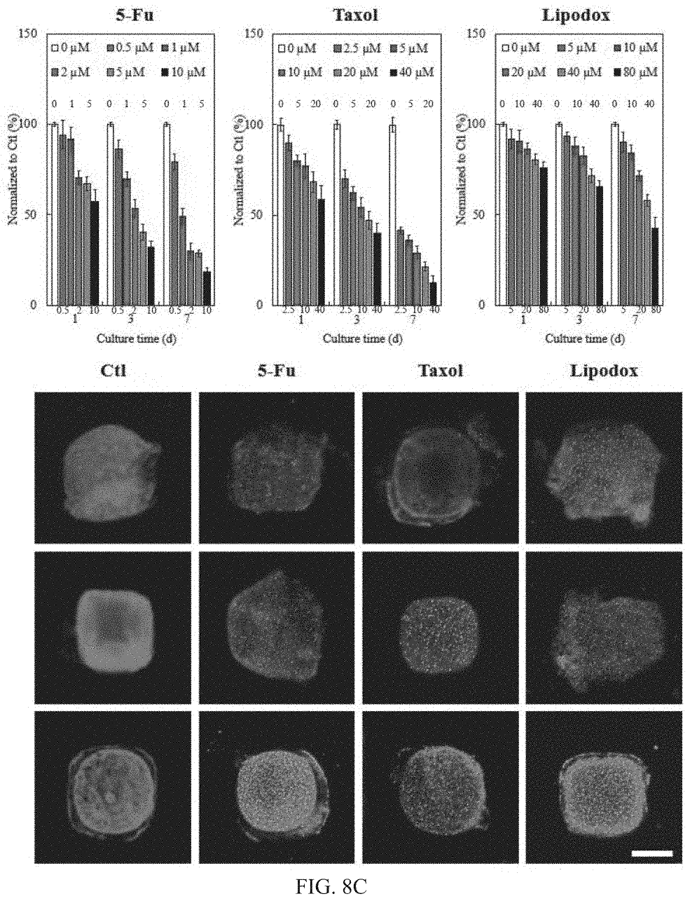

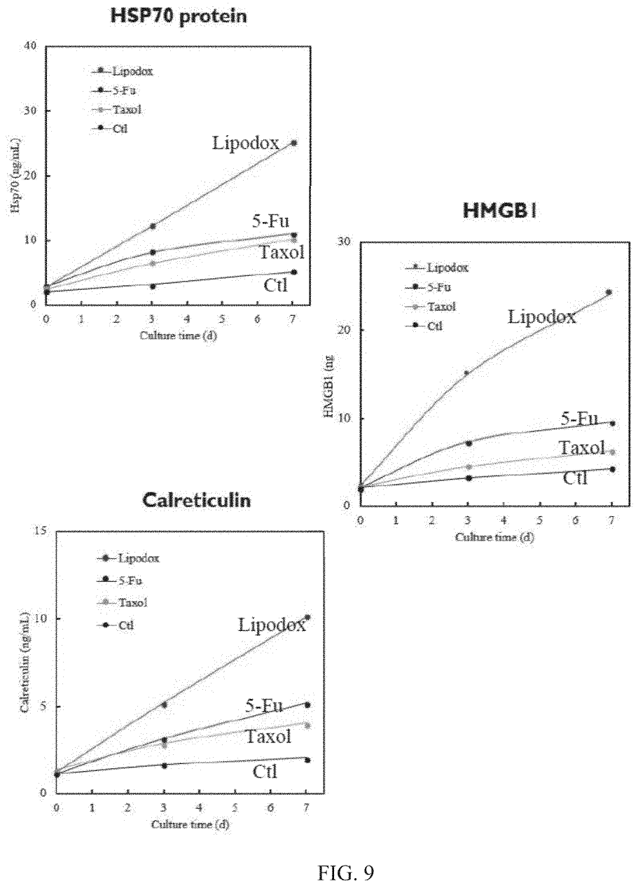

FIG. 9 is three different protein indicators extracted from the cancer cell metabolic waste fluid perfused by the biochip 10 of the present invention. These indicators including HSP70, HMGBI, and Calreticulin can show if the cancer cells are actually apoptotic. The present invention could estimate the condition of the cancer cells by analyzing its metabolic waste without sacrificing the cultivating cancer cells by taking them from the cultured condition from the biochip 10. The present invention could cultivate cancer cells for a long time.

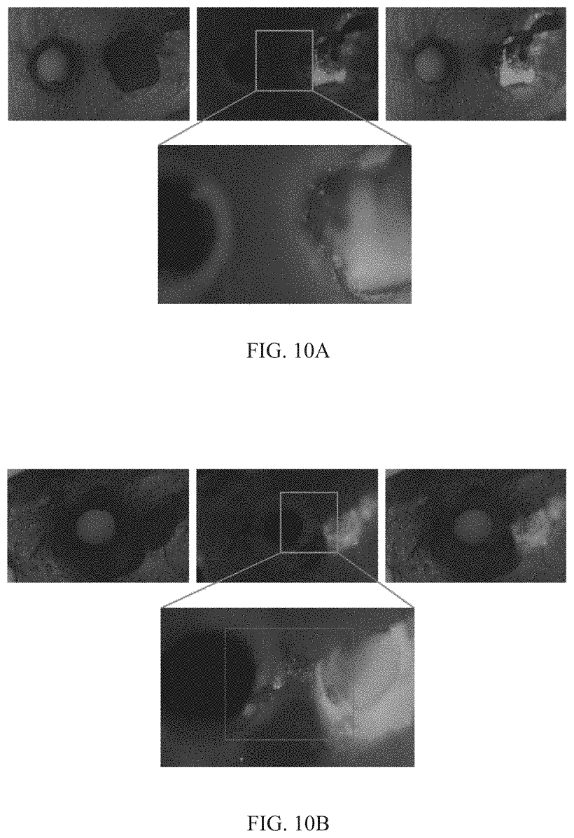

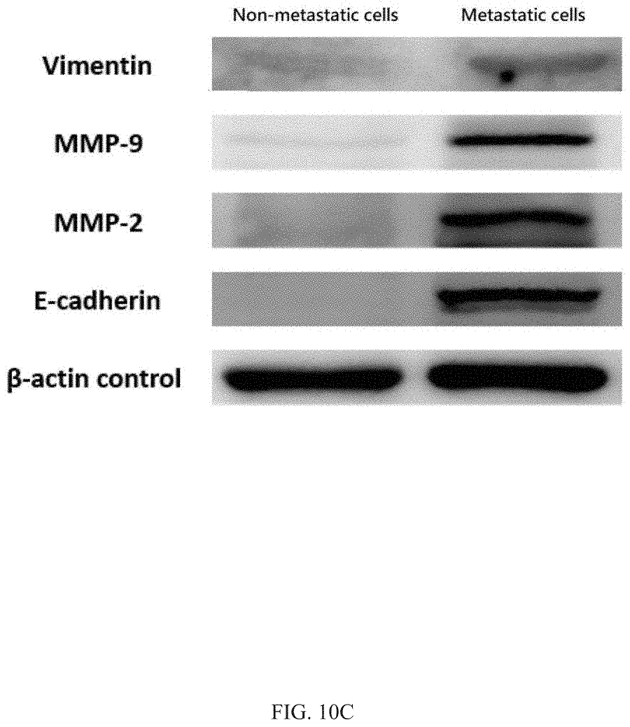

With reference to FIGS. 10A to 10B, the present invention could also use to detect if the tested cancer cell is a metastasis cancer. As shown in FIG. 10 A, it is an early stage of culture the said cancer cell on the biochip 10 of the present invention. On the right side, the cancer cell presented in a round shape. The hole on the right side is the simulated vascular channel. At this stage, there is no sign of migration of cancer cells. Further shown in the FIG. 10B, after a period of time of culturing, the bright spots of cancer cells gradually move towards the simulated vascular channel indicating that the cancer cells have the possibility of metastasis.

FIG. 10C is to confirm whether the cancer cells have metastasized by using several indicators that cancer metastasis will produce, including Vimentin, MMP-9, MMP-2, and E-cadherin by checking these indicators distribution condition in the perfusion simulated vascular channel from the biochip 10. The β-actin indicator is a control group of a cellular protein produced by all kinds of cells including normal cells and cancer cells. From the non-metastatic cells on the left side of FIG. 10C, it can be seen that there is no distribution of these indicators in the blood vessels indicating that the cancer cells have not metastasized. The cancer cells on the right side that have metastasized can clearly see the distribution of these index components in perfusion vessels. β-actin indicator however shows in both conditions indicating the cancer cells in biochips 10 of the present invention maintain alive and functioning.

<Lung Cancer>

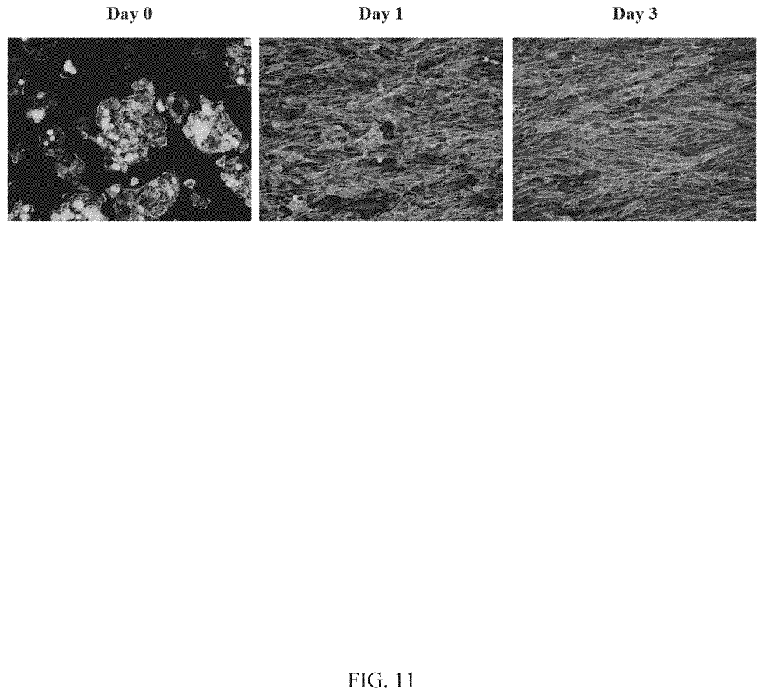

A validation result of a lung cancer using the biochip provided by the present invention shows in FIG. 11. As shown in FIG. 11, by culturing the cancer cell using dynamic perfusion, the growth of lung cancer cells is shown orthotropic which is more similar to the actual condition in the human body.

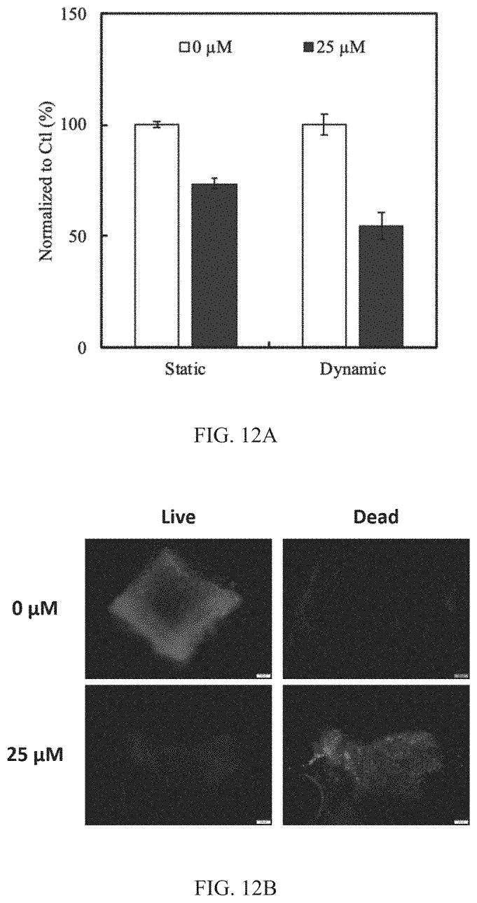

With reference to FIGS. 12A and 12B, cytotoxicity tests with 5-Fu cancer cell therapeutic drug are presented. It can be shown in FIG. 12A that the cytotoxicity of dynamic perfusion is more remarkable indicating that the therapeutic drug can be used under the dynamic culture of perfusion with better efficiency of killing cancer cells. FIG. 12B is an immunohistochemistry corresponding to FIG. 12A in which dead cancer cells are increased by using high dose of 5-Fu cancer cell therapeutic drug under the dynamic culture of perfusion.

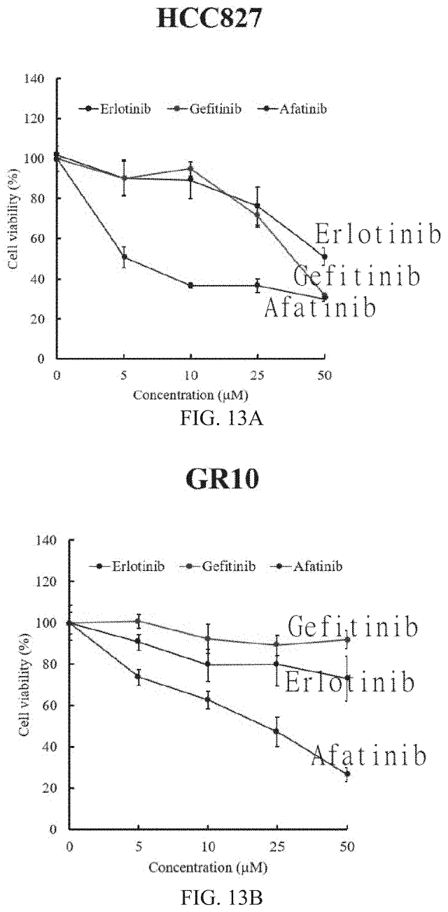

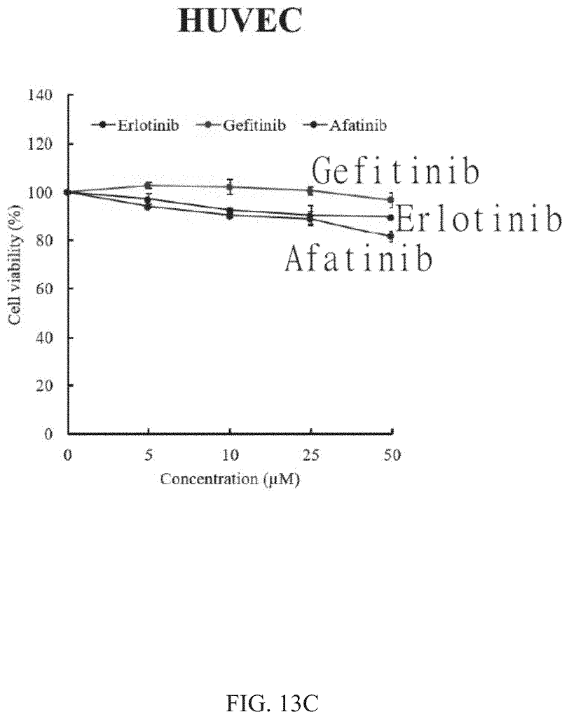

Please refer to FIG. 13A to FIG. 13C, wherein FIG. 13A and FIG. 13B are the cancer cell survival tests of three different cancer target drugs Erlotinib, Gefitinib and Afatinib using two different lung cancer cells HCC827 and GR10, and FIG. 13C Ordinary vascular cells were used as the control group. It can be seen from FIG. 13A and FIG. 13B that two different cancer cells have different cancer cell survival rates for three different target drugs. Among them, afatinib drug has a better effect on two cancer cells, and at high concentrations with the survival rate of cancer cells decreased significantly while Erlotinib and Gefitinib had significant effects on HCC827 lung cancer cells, but GR10 shows otherwise drug resistance. FIG. 13 shows that the three target drugs have no effect on general vascular cells and will not kill normal human cells.

<Breast Cancer>

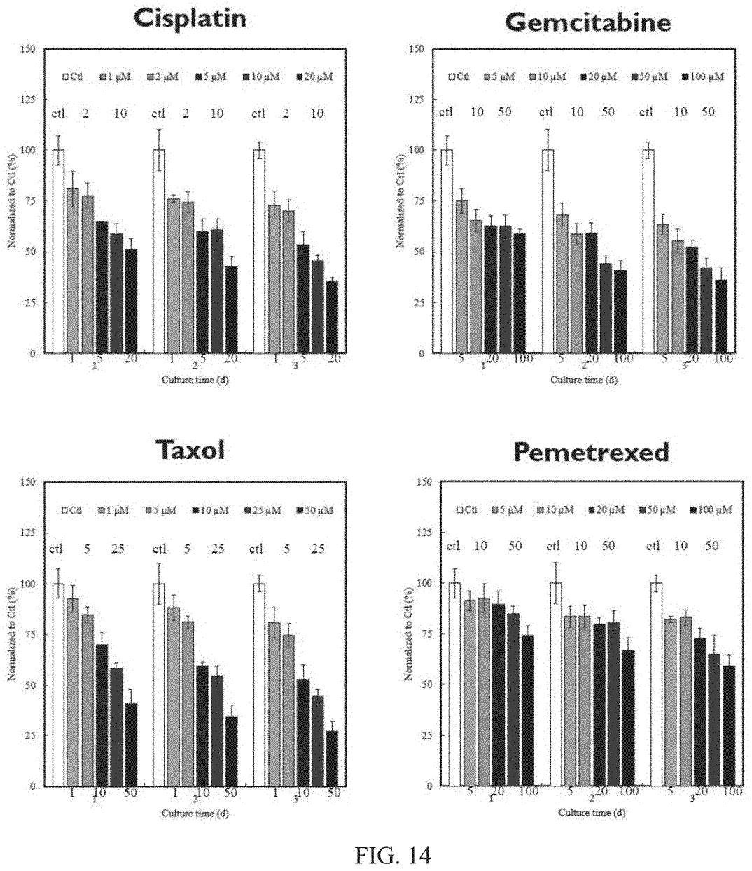

FIG. 14 is a validation test of cell viability of breast cancer cells treating by couples of cancer therapeutic drugs. As the result shown in FIG. 14, Taxol, Cisplatin and Gemcitabine show the ability of significantly reducing live cancer cells after culturing for 3 days in high concentrations. These three drugs show better reaction than Pemetrexed. By such, the present invention can be used to determine which cancer treatment drugs are effective for the patient.

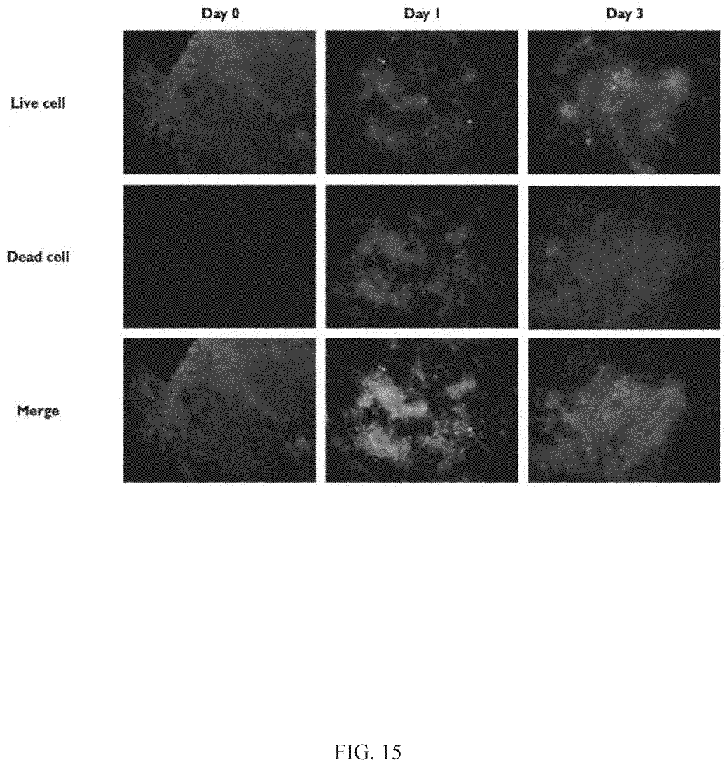

FIG. 15 is an immunohistochemistry corresponded to the test result of Taxol drug in FIG. 14. As shown in FIG. 15, more and more dead cancer cells are shown indicating the validation of the tested drug.

The above specification, examples, and data provide a complete description of the present disclosure and use of exemplary embodiments. Although various embodiments of the present disclosure have been described above with a certain degree of particularity, or with reference to one or more individual embodiments, those with ordinary skill in the art could make numerous alterations or modifications to the disclosed embodiments without departing from the spirit or scope of this disclosure.

What is claimed is:

1. A biochip comprising:
a carrier;
a cell or tissue culture area deposited on the carrier;
a sensor area deposited on the carrier adjacent and fluidly communicating with the cell or tissue culture area;
a containing space is contained in the cell or tissue culture area comprising a simulated vascular channel, a cell or a tissue and a culture medium; and
at least one sensor fixation area is contained at the sensor area for placing a sensor element.

2. The biochip as claimed in claim 1, wherein a wall is surrounded upwardly from a plane surface of the carrier to form the containing space.

3. The biochip as claimed in claim 2, wherein the wall comprises a supply port 115 and a discharge port 117 in fluid communication.

4. The biochip as claimed in claim 3, wherein the supply port and/or the discharge port include a supply port connecting portion and a discharge port connecting portion protruding inwardly or outwardly from the wall.

5. The biochip as claimed in claim 1, wherein the sensor area includes a fluid flow channel which fluid or liquid communicating with the at least one sensor fixation area.

6. The biochip as claimed in claim 1, wherein an air flow channel is further comprises in the containing space.

7. The biochip as claimed in claim 2, wherein an air flow channel is further comprises in the containing space.

8. The biochip as claimed in claim 3, wherein an air flow channel is further comprises in the containing space.

9. The biochip as claimed in claim 4, wherein an air flow channel is further comprises in the containing space.

10. The biochip as claimed in claim 5, wherein an air flow channel is further comprises in the containing space.

11. The biochip as claimed in claim 1, wherein a fluid for fluidly communicating the sensor area and the cell or tissue culture area contains nutrients or drugs.

12. The biochip as claimed in claim 2, wherein a fluid for fluidly communicating the sensor area and the cell or tissue culture area contains nutrients or drugs.

13. The biochip as claimed in claim 3, wherein a fluid for fluidly communicating the sensor area and the cell or tissue culture area contains nutrients or drugs.

14. The biochip as claimed in claim 4, wherein a fluid for fluidly communicating the sensor area and the cell or tissue culture area contains nutrients or drugs.

15. The biochip as claimed in claim 5, wherein a fluid for fluidly communicating the sensor area and the cell or tissue culture area contains nutrients or drugs.

16. The biochip as claimed in claim 1, wherein the culture medium further comprises immune cells or fibroblasts.

17. The biochip as claimed in claim 2, wherein the culture medium further comprises immune cells or fibroblasts.

18. The biochip as claimed in claim 3, wherein the culture medium further comprises immune cells or fibroblasts.

19. A production method of a biochip comprising steps of:
printing the biochip as claimed in claim 3 by three-dimensional printing;
filling the culture medium in the containing space of the cell or tissue culture area by three-dimensional printing;
placing a tubular support produced by a solvent-soluble material on a surface of the culture medium, and the supply port and the discharging port is connected with the tubular support;
printing a vascular simulating material along a surface of the tubular support to form the simulated vascular channel and following with printing and distributing the cells and the culture medium in the rest of the containing space covering the simulated vascular channel; and
using a solvent able to dissolve the solvent-soluble material to wash off the tubular support for the containing space to obtain the biochip.

20. The production method as claimed in claim 19, wherein fluxing an endothelial cell into a channel that formed by washed tubular support to adhere, distribute and culture at an inner surface to form the simulated vascular channel.
